(19) United States
(12) Patent Application Publication
(10) Pub. No.: US 2017/0273614 A1
(43) Pub. Date: Sep. 28, 2017

(54) SYSTEMS AND METHODS FOR MEASURING AND ASSESSING SPINE INSTABILITY

(71) Applicant: Halifax Biomedical Inc., Manou (CA)

(72) Inventors: Johan Erik GIPHART, Mabou, Nova Scotia (CA); Yann GAGNON, Mabou, Nova Scotia (CA); Chad MUNRO, Mabou, Nova Scotia (CA); Richard VAN DE PUT, Mabou, Nova Scotia (CA)

(21) Appl. No.: 15/505,014

(22) PCT Filed: Aug. 21, 2015
(86) PCT No.: PCT/CA2015/050805

§ 371 Date: Feb. 17, 2017

Related U.S. Application Data
(60) Provisional application No. 62/040,342, filed on Aug. 21, 2014.

(51) Int. Cl. A61B 5/00; A61B 6/03; A61B 6/04; A61B 5/107; G06T 7/00; A61B 34/10; A61B 6/02; G01R 33/56; G06T 7/73; A61B 6/00; A61B 5/11

(52) U.S. Cl. CPC A61B 5/4566; A61B 6/505; A61B 6/5217; A61B 6/032; A61B 6/04; A61B 5/702; A61B 5/1075; A61B 5/1128; A61B 5/1116; A61B 34/10; A61B 6/022; A61B 6/466; G01R 33/5608; G06T 7/75; G06T 7/0014; A61B 2034/105; A61B 2034/107; G06T 2207/10088; G06T 2207/30012

(57) ABSTRACT

Diagnostic systems and methods for measuring and assessing spine instability are described which involve reconstruction of a dynamic three-dimensional model of a patient's spine moving through a range of motions, and optimization of the three-dimensional model to provide relative three-dimensional position and orientation data for each vertebra in the spine throughout the motion. Vertebral movement is thereby accurately measured and instability determined for presentation in a user-friendly form.

### TECHNICAL FIELD

The present invention relates to the field of pre-operative diagnostics and, in particular, to pre-operative diagnostic systems and methods for measuring and assessing spine instability.

### BACKGROUND

Lower back pain (LBP) is one of the most prevalent causes of disability and interferes with the ability to work and decreases quality of life. Such pain is multifactorial and can result from a variety of spinal pathologies, however, spinal instability is considered to be a significant cause. Damage to the vertebral bodies, intervertebral discs, laminae, spinous processes, articular processes, or facets of one or more spinal vertebrae can result in the vertebrae no longer properly articulating or aligning with each other. When one spinal segment deteriorates in this way, to the point of instability, it can lead to localized or radicular pain, spinal stenosis, an undesired anatomy, and/or loss of mobility.

The widespread prevalence of LBP is reflected in the high cost to society in general, as well as high costs associated with treating LBP. It has been reported that the approximately 50 million patients suffering from LBP cost society in the United States a total of $240 billion annually. Of these costs, $50 billion is spent on spine surgery, and approximately $6 billion being directly attributable to diagnostics alone. Orthopaedic interventions, such as spinal fusion surgery, have become the standard of care in the United States, however, oftentimes such interventions have relatively poor outcomes and morbidity following failure is significant.

The ineffectiveness of current diagnostic methods, to identify proper candidates for these interventions, is to a significant amount responsible for the failure to successfully treat spinal instability. The lack of effective diagnostics has resulted in the absence of an agreed upon diagnostic standard and standard treatment protocols. For the most part, qualitative interpretation of radiologic tests are typically relied on to measure spinal instability. For example, X-rays of the spine in the neutral position (standing straight) and in flexion and extension are used to determine the amount of space between vertebrae and the condition of the vertebrae. A CT scan may further be used to get a better look at the vertebrae and facet joints including any bone spurs and small or complex fractures that may be present. In most cases, an MRI is also used to check for soft tissue lesions such as a herniated disc, degeneration or sites of inflammation. Static radiologic tests, used alone or in combination, are ineffective for assessing the movement of the spine throughout the entire range of motion. As such, current methods to diagnose and measure instability remain ineffective.

U.S. Pat. No. 8,676,293, describes an apparatus for positioning a patient through various joint motions in order to produce digital moving images of the joint motion. Electromyography is further combined in order to simultaneously produce data relating to muscle involvement associated with the specific types of joint motion. In this way, the process allows the relative motion, and associated muscle involvement, of certain skeletal structures of the patient to be measured. The diagnostic data that is produced, specifically two-dimensional linear and angle measurements, may be applied to generate clinically useful diagnostic data.

There continues to be a need for dynamic joint motion diagnostic methods that can provide the level of three-dimensional precision necessary for measuring spinal instability in a way that is clinically practicable and thus able to be integrated into a standard of care for spine instability diagnostics.

This background information is provided for the purpose of making known information believed by the applicant to be of possible relevance to the present invention. No admission is necessarily intended, nor should be construed, that any of the preceding information constitutes prior art against the present invention.

### SUMMARY

The embodiments of the present disclosure relate to systems and methods for measuring and assessing spine instability. In accordance with one aspect, there is described a diagnostic method for quantitatively measuring spinal instability in a patient, the method comprising: a) capturing a series of multi-frame stereo radiographic images of a target region of the patient's spine, wherein the patient is moving through a range of motion that allow for motion of vertebrae in the target region of the spine to be captured in the series of multi-frame stereo radiographic images; b) reconstructing a three-dimensional model of the target region of the patient's spine moving through the range of motion, wherein a relative three-dimensional position and orientation for each vertebra in the target region is calculated based on the radiographic images for each frame of the series of images; and c) measuring a change in the relative three-dimensional position and orientation of each vertebra in the three-dimensional model of the target region throughout the motion, wherein the measured change reflects the amount of spinal instability in the patient. According to certain embodiments, the method further comprises: d) displaying the change in the relative three-dimensional position and orientation of each vertebra as a three-dimensional movie. According to certain embodiments, the method further comprises: e) determining and analyzing the shape of the vertebrae.

In accordance with another aspect, there is described a method for assessing a patient's suitability for an orthopaedic procedure, the method comprising: a) capturing a series of multi-frame stereo radiographic images of a target region of the patient's spine, wherein the patient is moving through a range of motion that allow for motion of vertebrae in the target region of the spine to be captured in the series of multi-frame stereo radiographic images; b) reconstructing a three-dimensional model of the target region of the patient's spine moving through the range of motion, wherein a relative three-dimensional position and orientation for each vertebra in the target region is calculated based on the radiographic images for each frame of the series of images; c) measuring a change in the relative three-dimensional position and orientation of each vertebra in the three-dimensional model of the target region throughout the motion; and d) comparing the measured change in the three-dimensional model to instability data standards for normative and varying levels of instability, wherein the comparison indicates the degree of instability and the patient's suitability for an orthopaedic procedure. According to certain embodiments, the method further comprises: e) classifying the measured change by type and degree of instability of the vertebrae to determine the suitability of the patient for the orthopaedic procedure. According to certain embodiments, the method further comprises determining the shape of the vertebrae and comparing the shape of the vertebrae to normative shapes and shapes of patients with spinal pathology, wherein the comparison indicates the degree of pathology and the patient's suitability for an orthopaedic procedure. In such embodiments, the shape of the vertebrae can be classified by type and degree of pathology associated with LBP and/or spinal instability. According to other embodiments, the method is for assessing a patient's suitability for spinal fusion, artificial disk replacement, dynamic stabilization procedures, or conservative treatment, among other treatments.

In accordance with a further aspect, there is described a radiographic imaging method for generating a three-dimensional reconstruction of the movement of a target region of a patient's spine, the method comprising: a) capturing a series of multi-frame radiographic images of the target region of the patient's spine, the radiographic images comprising a pair of images taken at an angle of each other to capture images within a viewing volume wherein the patient is moving through a range of motion; b) calculating foci and edge data of vertebrae captured in a radiographic image in the series and consolidating the data to a common reference frame; c) determining a general three-dimensional position and orientation of the vertebrae; d) iteratively manipulating the general three-dimensional position and orientation of the vertebrae against the data in the common reference frame to achieve a best-fit three-dimensional position and orientation for each vertebra in the radiographic image; and e) repeating steps b to d for each image pair of a series; wherein a three-dimensional model of the target region of the patient's spine moving through the range of motion is generated. According to certain embodiments, step (c) of the method involves using a model encapsulating anatomical variability of a population, such as a statistical shape model to also iteratively determine the shapes of the vertebrae. According to other embodiments, step (c) of the method involves a three-dimensional model of the patient's vertebral spine derived from, for example, from a CT-scan or an MRI of the patient's spine.

According to another aspect, there is described a positioning apparatus for maintaining the position of a patient in a viewing area during radiographic imaging throughout a series of patient movements, for example lumbar flexion and extension, the apparatus comprising: a base for supporting a foot platform on which the patient stands when in position for radiographic imaging, the foot platform having a front end and a rear end; and a pelvic support extending from the base above the foot platform at the rear end, the pelvic support configured to support the patient's pelvis. According to certain embodiments, the positioning apparatus further comprises a knee support extending from the base above the foot platform at the front end, the knee support configured to support the patient's knees when the patient is positioned with ankles, knees and hips flexed.

### BRIEF DESCRIPTION OF THE DRAWINGS

These and other features of the invention will become more apparent in the following detailed description in which reference is made to the appended drawings.

FIG. 1 is a schematic illustration of a dynamic stereo radiography system in a 60 degree inter-beam configuration that may be used in an exemplary method, according to an embodiment of the present disclosure;

FIG. 2(A) is a schematic illustration of a pair of overlapping X-ray beams emitted from a dynamic stereo radiography system with a 90 degree inter-beam configuration, and the three-dimensional viewing volume generated by the overlapping beams, according to an exemplary embodiment of the present disclosure, while FIG. 2(B) is the schematic illustration of FIG. 2(A) shown together with an exemplary dynamic stereo radiography system disclosed herein;

FIG. 3 is a schematic illustration of an image registration and creation of a common reference frame (coordinate system) based on the sets of markers provided by the reference box of the exemplary dynamic stereo radiography system;

FIG. 4(A) is a schematic illustration of an exemplary positioning apparatus according to an embodiment of the present disclosure, while FIG. 4(B) is a schematic illustration of the positioning apparatus shown in FIG. 4(A), in operation;

FIG. 5 is a flowchart illustrating an exemplary radiographic imaging method for generating a three-dimensional reconstruction of the shape and movement of a target region of a patient's spine, according to an embodiment of the present disclosure;

FIG. 6 is a flowchart illustrating an exemplary process for generating a statistical shape model (SSM) for three-dimensional reconstruction of the shape and movement of a target region, according to an embodiment of the present disclosure;

FIG. 7 is a display illustrating bone-fitting tracking between a three-dimensional model and a pair of radiographic images to optimize shape, position and orientation for each vertebra in a target region in a first position of motion, according to an exemplary embodiment of the present disclosure;

FIG. 8 is a display illustrating bone-fitting tracking between the three-dimensional model shown in FIG. 7 in a second position of motion, according to an exemplary embodiment of the present disclosure;

FIG. 9 is an exemplary three-dimensional presentation of a patient's vertebral instability as determined by an exemplary method disclosed herein;

FIG. 10 is a schematic presentation of the combination of multiple variables reflecting a patient's vertebral instability into an instability score which maximally discriminates between healthy spine motion and unstable spine motion as determined by the exemplary methods disclosed herein;

FIG. 11 is another exemplary presentation of a patient's vertebral instability as determined by the exemplary methods disclosed herein;

FIG. 12 is a flow chart illustrating application of the exemplary radiographic imaging method shown in FIG. 5 for generating a three-dimensional reconstruction of the shape and movement of a target region of a patient's spine as outlined in Example 2 using a statistical shape model;

FIG. 13 is a flow chart illustrating application of the exemplary radiographic imaging method shown in FIG. 5 for generating a three-dimensional reconstruction of the shape and movement of a target region of a patient's spine as outlined in Example 2 using a 3D CT-scan model; and

FIG. 14 is a flow chart illustrating application of the exemplary radiographic imaging method shown in FIG. 5 for generating a three-dimensional reconstruction of the shape and movement of a target region of a patient's spine as outlined in Example 2 using a 3D model.

### DETAILED DESCRIPTION OF THE INVENTION

Diagnosis of spinal instability is routinely based on established static imaging methods, however, there is no single imaging modality to date which discriminates with sufficient certainty “normal” and “abnormal” motion. Imaging-based methods, therefore, are generally considered to be ineffective in the diagnosis of instability.

The embodiments of the present disclosure describe stereo imaging-based methods that allow instability of a patient's spine to be quantitatively assessed in 3D, multiple times per second while the patient is in a loaded or unloaded state. Specifically, the embodiments of the present disclosure include diagnostic methods for quantitatively measuring spinal instability based on reconstruction of a three-dimensional model of the patient's spine moving through a range of motion. Optimization of the three-dimensional model, provides shape and relative three-dimensional position and orientation data for each vertebra in the spine throughout the motion. From this relative data, the vertebral movement can be accurately measured and instability can thereby be quantitatively assessed.

According to certain embodiments, the present disclosure describes methods in which the vertebral movement of a patient's spine is presented in a user-friendly display having quantitative information overlaid for easy interpretation by the user. Such embodiments offer the user methods for assessing a patient's suitability for an orthopaedic procedure that is easy to understand without necessarily requiring qualitative interpretation of the images by a specialist such as a radiologist or an orthopaedic surgeon. According to embodiments described herein, methods for assessing a patient's suitability for an orthopaedic procedure involve comparing the measured change in the reconstructed three-dimensional model, described herein, to instability data standards for normative and varying levels of instability, wherein the comparison indicates the degree of instability and the patient's suitability for an orthopaedic procedure. According to further embodiments, the degree of instability and, hence the patient's suitability for an orthopaedic procedure, can be displayed in a user-friendly presentation for the user to quickly determine the suitability of the patient for an orthopaedic procedure. According to certain embodiments, the presentation can be displayed in a variety of formats and is adaptable to various vehicles such as a mobile phone, tablet, or laptop.

Unless defined otherwise, all technical and scientific terms used herein have the same meaning as commonly understood by one of ordinary skill in the art to which this invention belongs.

As used herein, the term “x-ray” and “radiographic imaging” are used interchangeably throughout the application to mean the same thing.

As used herein, the term “about” refers to an approximately +/−10% variation from a given value. It is to be understood that such a variation is always included in any given value provided herein, whether or not it is specifically referred to.

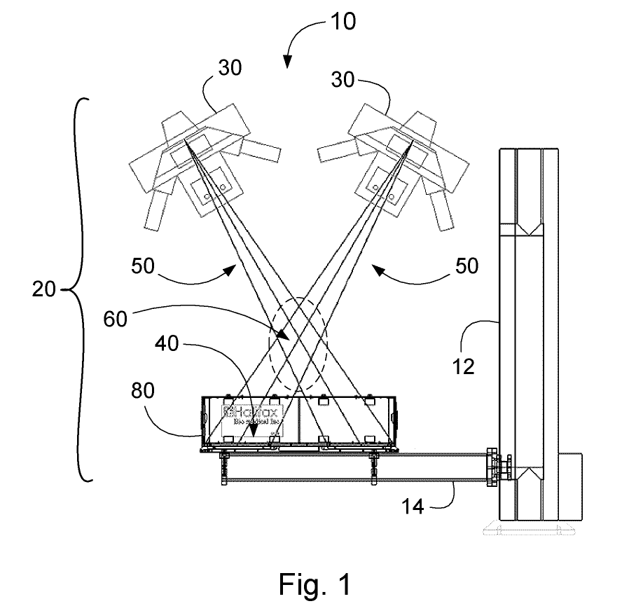

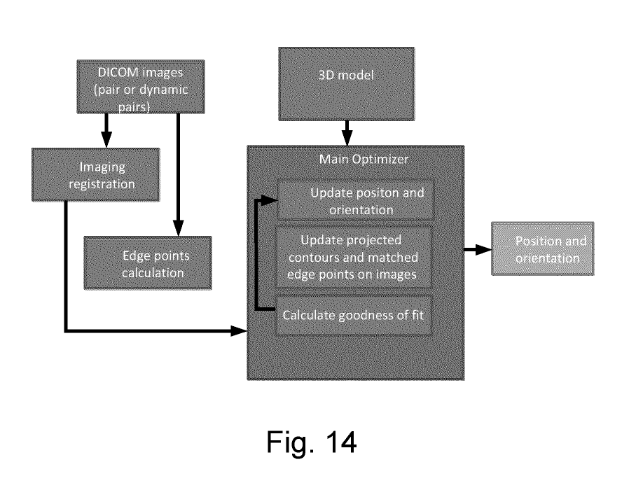

For purposes of illustration, the devices and methods of the invention are described below with reference to the spine of the human body. However, as will be appreciated by those skilled in the art, the devices and methods can be employed with any mammal and for any joint. Embodiments of the present disclosure will now be described by reference to FIGS. 1 to 14.

### Stereo Dynamic X-Ray Imaging

A feature of the embodiments of the present disclosure relates to the 3D reconstruction of shape, position, and orientation of the vertebrae in a patient's spine. Specifically, a three-dimensional reconstruction of the movement of the spine is generated and optimized based on a series of multi-frame radiographic images of the patient's spine. From this optimized dynamic three-dimensional model, the 3D micro stability of the spine can be measured. Persons of skill in the art will recognize that a series of progressive static radiographic images may be used to generate multi-frame radiographic images.

Persons of skill in the art will recognize that there are a variety of imaging and reconstruction methods that may be used to generate the three-dimensional model of the spine. For example, biplane or dual-plane fluoroscopy may be an alternative imaging technology, or dynamic radiostereometric analysis (RSA) may be an alternative reconstruction method. Without limiting the foregoing, certain embodiments of the present disclosure relate to a radiographic imaging method for generating a three-dimensional reconstruction of the movement of a target region of a patient's spine that comprises capturing a series of multi-frame stereo x-ray exposures of a patient who is upright (loaded position) or lying on a table (unloaded supine position). According to further embodiments, as is readily understood by those skilled in the art, weights, rubber bands, etc., can further be used to load the spine.

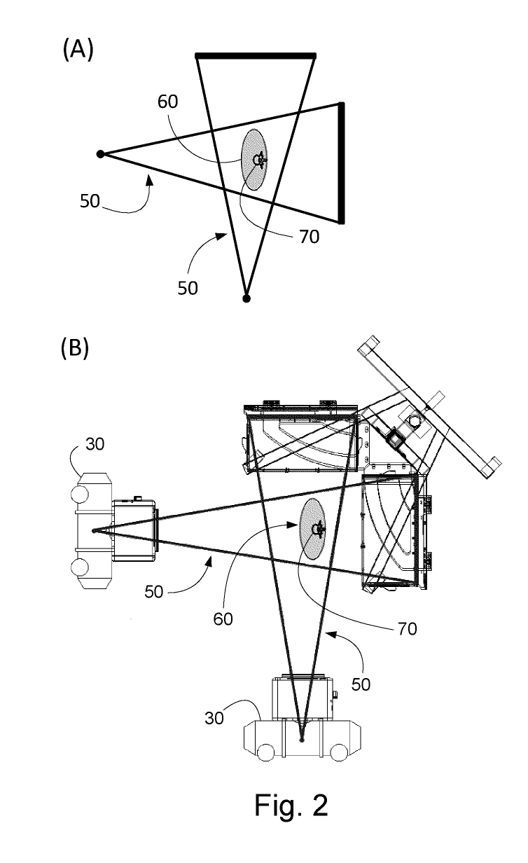

Referring to FIGS. 1 and 2, an exemplary dynamic stereo radiography system 10 that may be used in the 3D reconstruction of the presently described methods is illustrated. According to embodiments of the present disclosure, the stereo radiography system 10 consists of at least two x-ray imaging systems 20 each consisting of an x-ray source 30 and an x-ray detector panel 40. Each x-ray source 30 may be rigidly or loosely connected to its corresponding x-ray detector panel 40. Both the x-ray source 30 and detector panel 40 are capable of emitting and receiving multiple exposures per second. According to certain embodiments, the x-ray imaging systems 20 are capable of emitting and receiving up to 30 images/sec. According to further embodiments, the x-ray imaging systems 20 are capable of emitting and receiving at least 4 images/sec. According to other embodiments, the x-ray imaging systems 20 are capable of emitting and receiving at least 10 images/sec.

The timing of each exposure is precisely controlled, for synchronous and asynchronous applications. According to certain embodiments, the exposures are accurately synchronized such that both x-ray systems 20 are imaging at the same time. According to embodiments, short exposures are desirable to minimize motion blurring. The two x-ray imaging systems 20 are positioned at an angle to each other such that the x-ray beams 50 overlap in part to create a 3D viewing volume 60. In operation, the target region 70 of the patient's spine is positioned and maintained within this 3D viewing volume 60 throughout the series of exposures of a given range of patient motion. In this way, a dynamic multi-frame series of images may be captured. Persons of skill in the art may recognize that x-ray exposures may also be alternated as long as the timing is accurately controlled and known.

The 3D viewing volume 60, corresponding to the volume of the overlapping beams, provides the accuracy in the 3D reconstruction capabilities of the system 10. According to embodiments of the present disclosure, the angle between the two x-ray systems 20 is up to about 45 degrees. According to certain embodiments, the angle between the two x-ray systems 20 is up to about 60 degrees. According to other embodiments, the angle between the two x-ray systems 20 is up to about 90 degrees. According to further embodiments, the angle between the two x-ray systems 20 is at least about 60 degrees. According to preferred embodiments, the angle between the two x-ray systems 20 is about 90 degrees.

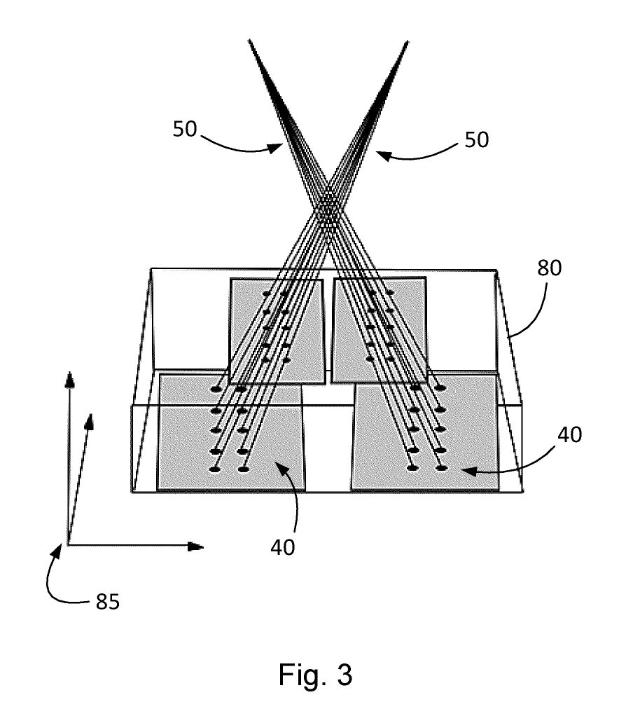

The dynamic stereo radiography system 10 also includes a reference box 80 (FIGS. 1, 3) which for each x-ray detector panel 40 provides two sets of markers. The fiducial set of markers located close to the detector panel 40 provides the analysis software with a coordinate frame 85, a linear scale, and allows for image distortion correction. The control set of markers located more towards the x-ray source 30 allows for the determination of the focus position of the x-ray source. The reference box 80 is typically rigidly constructed and the 3D positions of the makers are known. The reference box 80 is securely mounted onto a beam 14 that is pivotably engaged with a vertical support column 12 whereby the beam 14 can be controllably raised upward and downward and additionally controllably rotated vertical support column 12. A table (not shown) may be demountably engaged with the beam 12 and/or the reference box 80 so that a patient may be positioned underneath the x-ray sources 30 in a supine position, a prone position, or lying on either side. The beam 12 and the x-ray sources 30 may also be pivoted 90° so that the images of the patient can be recorded while they are standing, sitting, squatting and the like.

In order to ensure accuracy in the series of multi-frame images, the target region 70 of the patient's spine must be positioned and maintained within the 3D viewing volume 60 throughout the series of exposures of a given range of patient motion. In this way, a dynamic multi-frame series of images may be captured.

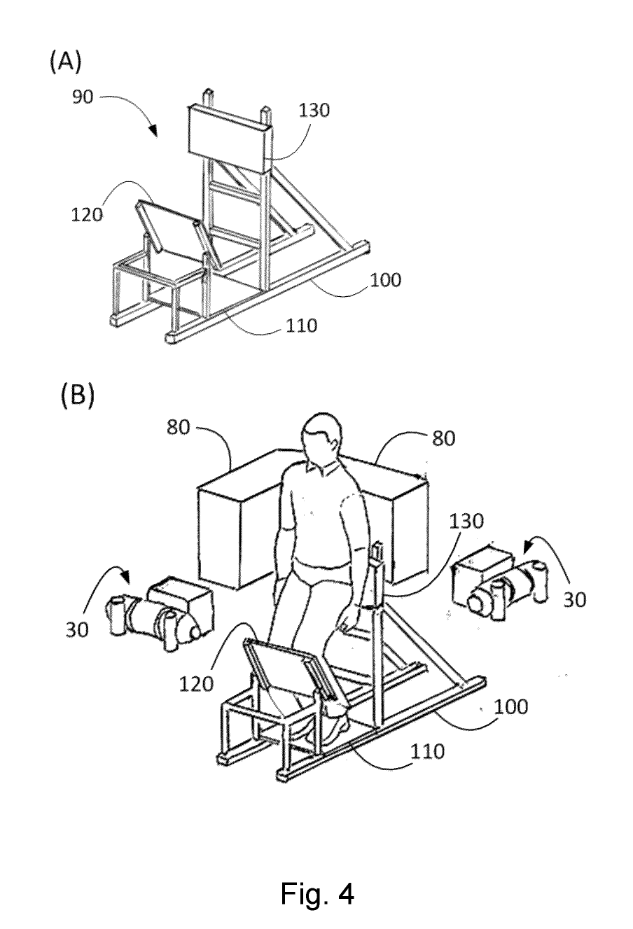

According to certain embodiments, a positioning apparatus is used to maintain the position of a patient in the 3D viewing area 60 while allowing the patient to move freely in a supported manner during radiographic imaging throughout a series of patient movements. Referring to FIGS. 4(A) and 4(B), the apparatus 90 according to preferred embodiments comprises a base 100 for supporting a foot platform 110 on which the patient stands when in position for radiographic imaging. A knee support 120 extends from the base 100 above the foot platform 110 at the front end. The knee support 120 is configured to support the patient's knees when the patient is positioned with ankles and knees flexed. A pelvic support 130 extends from the base 100 above the foot platform 110 at the rear end, and is configured to support the patient's pelvis when the patient is positioned with hips flexed. The apparatus 90 allows the patient to rest with their ankles, knees and hips flexed while preventing parasitic movements (e.g., hip flexion and extension) during lumbar flexion and extension. According to embodiments of the present disclosure, the various components of the positioning apparatus 90 are adjustable to accommodate patients of various sizes. For example, the base 100, foot platform 110, knee support 120, and/or pelvic support 130 can each be independently adjustable to accommodate height and angle of a patient. According to alternative embodiments, the knee support 120 may be omitted and patient may stand upright against the pelvic support. In yet another embodiment a restraining pad in front or on the sides of the patient attached to the pelvic support may be used for additional stabilization of the patient's pelvis. In yet another embodiment, hand grips may be provided for steadying the patient while entering the device or performing the motions.

### 3D Reconstruction of Shape, Position, and Orientation of Vertebrae

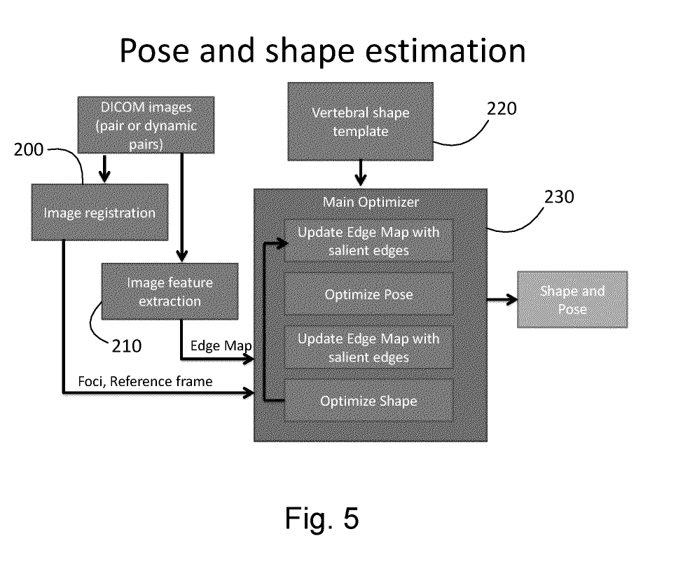

The three-dimensional reconstruction of the movement of a patient's spine consists of establishing a geometric relation between the vertebral representation in the stereo radiographic images and a 3D model of the patient's spine. According to embodiments of the present disclosure, methods for the 3D reconstruction involves fitting a vertebral shape template to foci and edge or gradient data of the patient's corresponding vertebrae captured in the radiographic images (FIG. 5). In this way, the shape template is optimized to best-fit the vertebral position and orientation derived from the radiographic images of the patient's spine. By calculating such optimization for each frame in a series of radiographic images, an optimized dynamic three-dimensional model is generated from which the 3D micro stability of the spine can be measured.

As described, image registration 200 (FIG. 5) of the radiographic images involves determining x-ray foci from the series of multi-frame stereo radiographic images and consolidating all image information into a common reference frame 85. According to embodiments of the present disclosure, a registration element exemplified by the reference box 80 shown in the apparatus 10 illustrated in FIGS. 1-3, is positioned between the patient and the detector panels 40. The registration element has a series of fiducial and control beads that provide reference markers from which x-ray foci can be calculated and all image information can be consolidated in a common reference frame 85 (FIG. 3).

Image feature extraction 210, according to embodiments of the present disclosure, includes filtering of the images for improved image quality and advanced gradient calculations, the robust detection of edges in the images, and the creation of a dynamic edge map.

The vertebral shape template 220 can be generated using a variety of methods known to those skilled in the art. According to embodiments of the present disclosure, the vertebral shape template can be derived from a CT-scan or MRI, or other patient-specific 3D imaging of the patient's spine. According to other embodiments, the vertebral shape template can be derived from population data to generate a shape model that encapsulates the anatomical variations among a population. This includes, but is not limited to, statistical shape models, statistical appearance models, statistical bone density models, parameterized shape models, or population atlases.

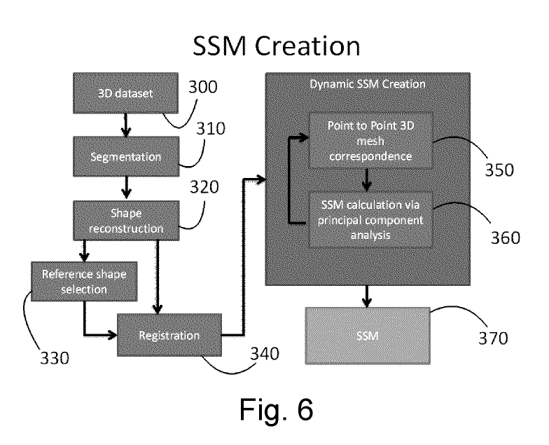

Statistical shape models (SSM) use principal component analysis to separate a set of shapes from a population into an average shape and a set of orthogonal shape variations (called modes) that behave much like a mean and a multidimensional set of standard deviations. Each shape can then be represented by a greatly reduced set of numbers describing how much of each anatomical variation (mode) is present in this particular shape. Moreover, it is quite common that an even more limited set of modes accounts for the vast majority of shapes, reducing the set of numbers needed to describe the shape even further. The general process for generating an SSM, according to embodiments of the present disclosure, is shown in FIG. 6. A set of shapes representative of a certain population is first created 300. According to embodiments of the current disclosure, these shapes can be derived from CT scans or other 3D imaging set by selecting the bone in each image i.e. by segmentation 310, and then reconstructing the shape from the 3D segmentation volume 320. A reference shape 330 is then selected to which all other shapes are referenced i.e. registered 340. Point to point correspondence is determined between each shape of the set 350 and the reference shape, and principal component analysis (PCA) is then performed 360. In certain embodiments, the SSM can then be used as the new reference shape and the process can be repeated (dynamic SSM) to improve the point-to-point correspondence among the shapes. The resulting output is a statistical shape model 370 able to represent the population shapes in the learning set as well as all other intermediate shapes not present in the learning set. In this way, a dynamic 3D vertebral shape template is generated.

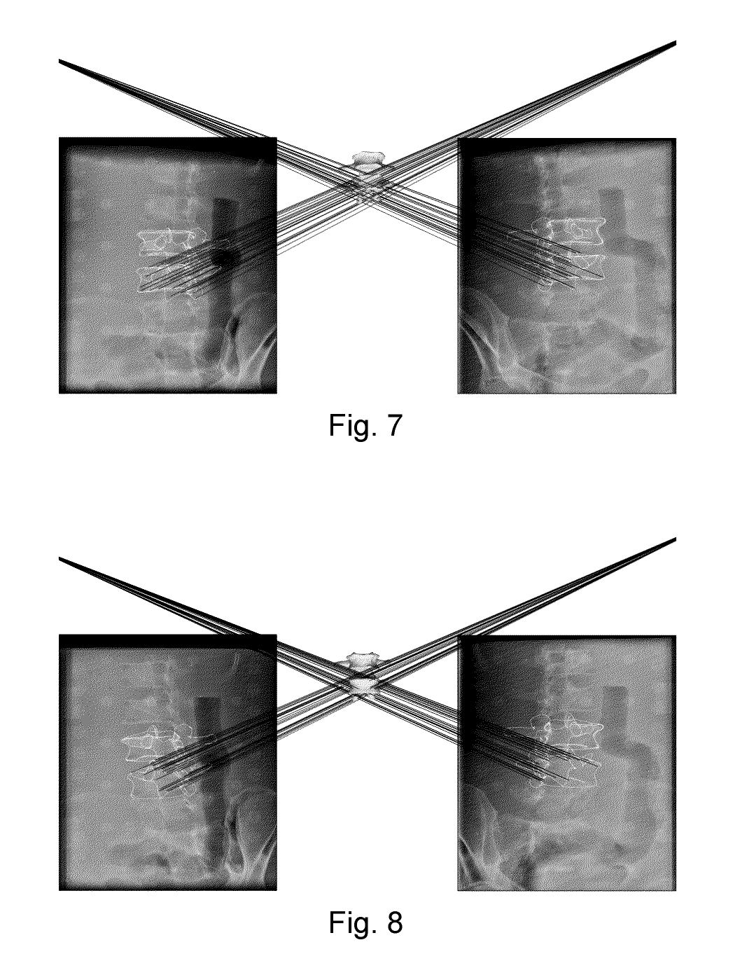

Referring to FIG. 5, the main optimizer 230 then involves iteratively fitting the general three-dimensional position and orientation of the vertebrae of the generated 3D vertebral shape template to the edge or gradient data in the common reference frame to achieve a best-fit three-dimensional position and orientation for each vertebra (FIGS. 7 and 8). Depending on whether the vertebral shape template is patient specific or population based, the iterations involve optimizing the shape within the constraints of the template. Moreover, edge data from the edge map may updated based on goodness of fit with projected vertebral models as well. The steps in the main optimizer are repeated for each image pair of a series of radiographic images to create an optimized three-dimensional model of the target region of the patient's spine moving through the range of motion. The resulting output is the shape of each of the vertebrae and the sacrum, and the 6 degree-of-freedom (DOF) orientation (pose) (i.e., three positions, e.g., X, Y, Z, and three rotations) of each vertebra relative to the other. The pose will be most relevant between adjacent vertebrae and traditionally the pose of a vertebra is described relative to the vertebra directly below.

### Measuring Multi-Frame Motion Between Vertebrae

According to embodiments of the present disclosure, the optimized dynamic three-dimensional model provides an accurate representation of the target region of the patient's spine moving through a range of motion to enable quantitative measurements to be determined. In particular, a change in the relative three-dimensional position and orientation of each vertebra in the three-dimensional model of the target region throughout a motion can be measured, reflecting the amount of spinal instability in the patient. According to particular embodiments, the change in the relative three-dimensional position and orientation of each vertebra can be presented as a three-dimensional movie to show the patient's 3D motion of the spine during the imaging exercise. According to some embodiments, the change in the relative three-dimensional position and orientation of each vertebra may be normalized relative to the relative 3D position and orientation of the other vertebrae of the patient's spine.

### Comparative Quantitative Identification of Spinal Instability

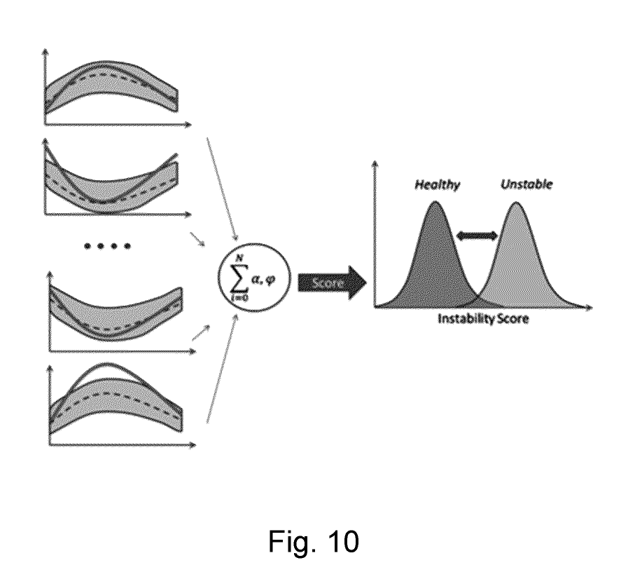

According to embodiments of the present disclosure, the measured change derived from the three-dimensional model can be applied as a diagnostic. According to such embodiments, the 3D measurements of two vertebrae derived from the optimized 3D model, is compared to instability data standards for normative (i.e., measurements taken from healthy people) and varying levels of instability (i.e., measurements taken from patients with (lumbar spine) instability). According to embodiments of the present disclosure, the instability measures at one particular spinal level may also be compared to the other (healthy) spinal levels within the same patient to determine the varying levels of instability. Based on multivariate or discriminant analyses (or similar techniques known in the art), the variables that are most able to separate the healthy and unstable joints are selected. These variables are then used to most optimally separate the two groups and to generate a spine instability score. In addition, gradations between healthy and unstable spines can be developed based on this instability score. Multiple instability types may become apparent and scores related to each type and an aggregate score may further be developed (FIG. 10). In this respect, scores may further be calculated to classify the type and quantify the degree of instability. These scores may be used directly for clinical decision making such as in treatment selection or the decision whether to go forward with a particular treatment as well as to enhance the data presentation in a resulting report.

### Instability Data Presentation

The descriptive data of the spine motion will contain a large number of variables that will change over time during a given motion. Such data is complicated and requires specialist expertise in order to decipher diagnostic meaning from the data. For example, specialized knowledge is required to fully understand the complicated set of motion values and scores as well as their respective diagnostic thresholds and instability severity grades. Methods of the present disclosure, however, offer a user interface that overlays quantitative information on top of a familiar qualitative presentation of the data to assist the physician in interpreting the results. According to certain embodiments, the user interface will focus and alert the physician to those portions of the data that are suggestive or indicative of pathology.

Specifically, a colour coding can be used in various display types that is uniform across the display types and indicative of the grade or severity of the clinical instability. According to exemplary embodiments, a colour coding scheme can be presented wherein Grade 0 indicates a healthy diagnosis represented by a Green colour code; Grade I indicates minor instability, represented by a Yellow colour code; Grade II indicates moderate instability, represented by an Orange colour code; and Grade III indicates severe instability, represented by a Red colour code. Other coding schemes can be utilized as will be apparent to those skilled in the art.

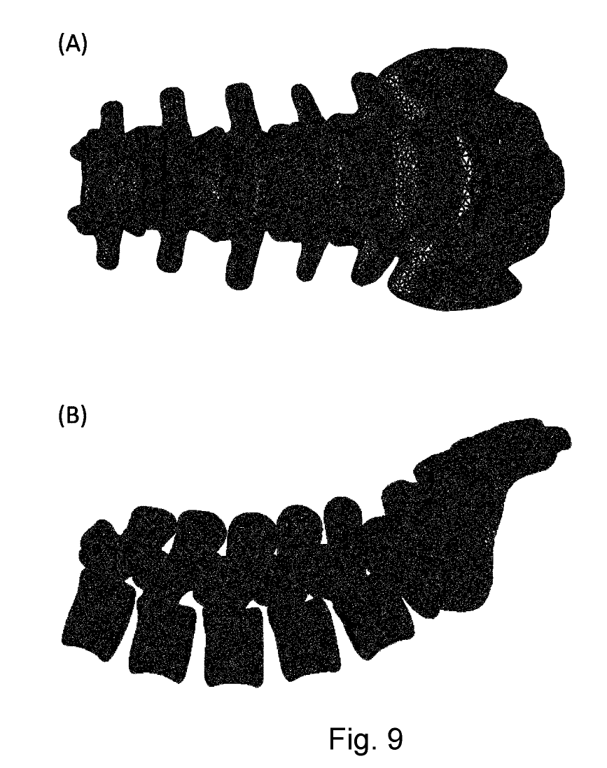

A number of display options are further contemplated. According to one embodiment, the type of instability may be exaggerated in a 3D movie display by de-emphasizing deviations from normal that are low risk and emphasizing deviations from normal that are high risk by using multiplication factors in the display of motion. Alternatively, the type and severity may be communicated through the addition of colour to the bones to show severity or type of instability. As illustrated in FIG. 9, 3D visualization of the spine motion is presented with vertebrae colour-coded based on their 3D motion data and/or instability score. For example, the vertebrae can be coded gray or green if no instability is detected. The frames for which the motion is outside the normal boundary, the vertebrae can be colour-coded yellow, orange or red depending on the extent of the severity of the instability. In such embodiments, it is contemplated that the 3D visualization can be a movie allowing the user to either rotate the spine to look at it from any desired angle, or multiple standardized views can be presented, with or without preset view buttons to easily switch between the preset views (e.g., anterior-posterior view, and lateral view).

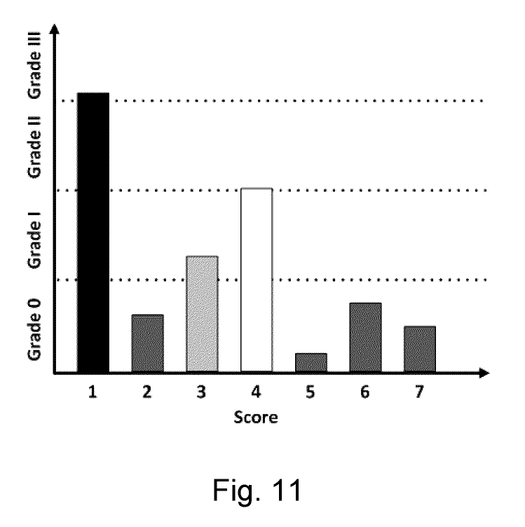

According to another embodiment, the visualization of variables or scores as dynamic bar graphs that move up and down during the motion is contemplated. In such an embodiment, the dynamic bars can be colour-coded based on the colour scheme described above and further depending on their magnitude (FIG. 11).

According to another embodiment, highlighted plots of variables are contemplated wherein the colour plots of variables can change over time depending on whether the variable exceeds the grade thresholds or not. The normal range for the variable may be displayed and a bar moving across the plot indicating the current time point may be displayed.

According to further embodiments, the presentation may be a combination of the above-described display types. All colour coding and time points in such an embodiment will be synchronized and animated between the display types.

### EXAMPLES

### Example 1

### Imaging Apparatus

Two separate radiography systems are used simultaneously to obtain stereo radiographic images. Each radiography system comprised an x-ray source (RAD-92 Sapphire X-Ray Tube; Varian Medical Systems, Palo Alto, Calif., USA), a generator (Hydravision SHF635RF DR X-Ray Generator, SEDECAL USA Inc., Buffalo Grove, Ill., USA), a digital imaging system (CDXI 50RF, Canon USA Inc., Melville, N.Y., USA), and a computer system to link the components together, to retrieve the imaging data, and to reconstruct the imaging data.

A 90-degree reference box (SR Reference Box; Halifax Biomedical Inc, Mabou, NS, Canada) was placed into the image field of both systems, as illustrated in FIG. 2. The reference box was constructed from carbon fiber to insure rigidity, to resist deformations resulting from temperature fluctuations during operation, and for its radiolucency. The reference box housed the detector panels in the back (away from the patient and x-ray source), immediately behind the fiducial planes which contained a series of equidistantly spaced radio opaque tantalum beads. The front of the box formed the control planes which contained radio-opaque tantalum beads also. The fiducial beads allowed the captured images to be transformed to a common reference frame, while the control beads allowed the calculation of the foci (i.e., the x-ray sources) locations to enable the analysis.

The images were captured on digital detector plates (CDXI 50RF; Canon USA Inc, Melville, N.Y., USA) as greyscale images with relative intensity values in standard medical DICOM format. The overlap of the two radiography systems' fields of view made up the 3D viewing volume.

### The Spine Positioning Device and Image Data Acquisition

In order to keep a patient's spine within the 3D viewing volume of the stereo radiography system during the imaging process, the patient was positioned in the positioning device (similar to that exemplified in FIGS. 4(A), 4(B). For some of the imaging sequences, the patient stood with their feet positioned toward the rear of the platform and with their pelvis rested against the pelvic support while a technician monitored their positioning, posture, and the patient moved from a neutral position to flexion then to extension and then back to the neutral position. For other imaging sequences, the patient additionally rested on the knee support while patient performed the movements. Each of the image sequence recordings was reviewed by the technologist to ensure image quality and the regions of interest were captured. The images were then transferred using tele-radiology technology to the image analysis center for analysis.

### System Configuration Determination

The radiographic images were loaded onto a computer system for calculation of the parameters that described the detailed configuration of the imaging system. The fiducial beads in the reference box were located in the images and their locations tabulated. Based on the known locations of these beads, a projective transformation was calculated that matched the bead locations to the tabulated locations from the images. The control beads of the reference box were located in the images and their locations tabulated. Based on the known locations of the fiduciary beads and the control beads, the locations of the two foci were calculated.

### Creation of a Statistical Shape Model

The statistical shape model was created based on CT datasets of adults following a process outlined in the exemplary flow chart shown in FIG. 6. The CT data was converted to 3D mesh models by segmentation of the bones by a trained user followed by 3D triangulation for all lumbar vertebrae using Mimics (Materialise NV, Leuven, Belgium). All 3D models were brought to a common alignment and location using an iterative closest point algorithm. Point-to-point correspondence was generated between all the 3D models using thin-plate-splines using an initial template mesh. Once all the models were in correspondence, an average collection of points was calculated, which was then triangulated with a ball pivoting algorithm, which generated the average 3D model. A principal component analysis was performed on the points in correspondence which calculated the principal modes of variation. These described the deviation of each point from the average 3D model. In this way, the statistical shape model has the following components: a triangulated mesh representing the average shape, an eigenvector matrix representing the principal modes of variation which can be multiplied by the average shape's vertices location to generate new shapes and a variance vector representing the variability of each mode of variation. Once an initial statistical shape model existed, the template mesh was replaced with meshes generated from the statistical shape model. This improved the point to point correspondence and allowed the calculation of an improved statistical shape model.

### Reconstruction of Vertebral Position, Orientation and Shape

A graphic user interface allowed the operator to manipulate the position, orientation and first three modes of the shape via sliders, and to immediately see the results of the projected contours onto the radiographic image. The location of the foci and the parameters describing the projective transform were used to calculate the projected contours onto the fiducial plane for any given position, orientation and shape of each vertebra. In this way, the operator set the initial position, orientation and first three modes of the shapes, which were saved and used as the starting points for the optimizer.

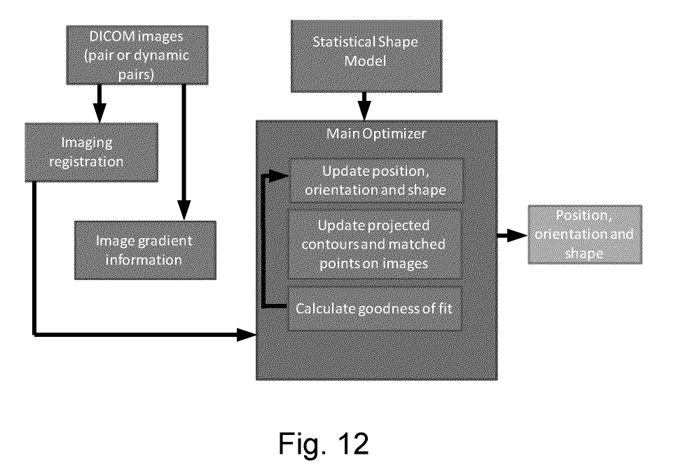

An objective function was made available to the optimizer which calculated a goodness-of-fit score between the projected contours and detected contours given a position, orientation and shape, generally following the process shown in FIG. 12. The detected contours were determined based on the gradient of the image. The goodness of fit score was based on the quality of the correspondence (the number of points suitably matched), and the sum of squared distances and direction match scores of the projected points.

The optimizer used the objective function to find the position, orientation and shape that provided the best fit to the radiographic images, within a predefined search space. The entire parameter space was searched in this example, which is to say that position, orientation and shape were all optimized simultaneously. In this example, the optimizer first used Particle Swarm Optimization as a global optimization method. A second round of optimization attempted to further increase the goodness-of-fit with a local-gradient-based optimizer. The initial position of the particles was normally distributed along the predefined search space and centered on the user initialized estimates. The optimizer returned the final position, orientation and shape of the 3D vertebra model.

In the same way, the final position, orientation and shape of the 3D vertebra was calculated for every set of images in a series. The optimizer assumed that the shape of the vertebra is the same in every image of the series. The optimizer used the position and orientation of a previous image in a series, combined with knowledge of the context of the acquisition to automatically initialize the position and orientation without user interaction. The vertebrae of the target region were reconstructed for the entire set of multi-frame radiographic images throughout the motion.

### Diagnostic Measurements

With the reconstructed vertebra models, position and orientation, the motion of each vertebra was described relative to a chosen reference point, which was the vertebrae below it (or sacrum in the case of L5). Based on the relative motion of each vertebra to its neighbours, measurements of clinical relevance to vertebral instability were calculated such as anterior translation, posterior/anterior rotation and the relative translation per degree of rotation were calculated for each spinal segment of interest. These measurements were compared to normative data to assist in assessing a patient's degree and type of spinal instability. The shape of the vertebra was also compared to normative data. In this case the statistical shape model provided the reference and each mode describing the shape was related to the degree of deviation from the normal, average shape. These morphological features were compared against known combinations, from normative data, which would predispose a vertebra to a pathological condition.

### Diagnostic Presentation and Clinical Decision

The diagnostic measurements were presented to the surgeon and patient using a visualization interface. The interface was web-browser based and available for viewing with proper credentials on any internet enabled device. All the measurements were made available for viewing, with the presentation depicting the relation of the patient's measures relative to normative data. The presentation was color coded to clearly present the deviation of the patient's diagnostic measurements in relation to the normative data. An aggregate score was calculated as a global indicator of instability for each spine segment of interest.

Based on the deviation from normal in both the motion and shape combined with the clinical evidence relating the abnormality found in this patient to good clinical outcomes from a spinal fusion surgery, the treating surgeon and patient decided to schedule the spinal fusion surgery.

### Example 2

A stereo orthopaedic radiography system (Halifax SR Suite; Halifax Biomedical Inc, Mabou, NS, Canada) was used consisting of two radiography systems exposing consecutively to obtain stereo radiographic images. Each radiography system comprised an x-ray source (RAD-92 Sapphire X-Ray Tube; Varian Medical Systems, Palo Alto, Calif., USA), a generator (Hydravision SHF635RF DR X-Ray Generator, SEDECAL USA Inc., Buffalo Grove, Ill., USA), a digital imaging system (CDXI 50RF, Canon USA Inc., Melville, N.Y., USA), and a computer system to link the components together, to retrieve the imaging data, and to reconstruct the imaging data.

A 60-degree reference box (SR Reference Box; Halifax Biomedical Inc, Mabou, NS, Canada) was placed into the image field of both systems, as illustrated in FIG. 1, The reference box was constructed from carbon fiber to insure rigidity, to resist deformations resulting from temperature fluctuations during operation, and for its radiolucency. The reference box housed the detector panels in the bottom (away from the patient and x-ray source) in a uniplanar configuration, immediately behind the fiducial planes which contained a series of equidistantly spaced radio opaque tantalum beads. The top of the box formed the control planes which contained radio-opaque tantalum beads also. The fiducial beads allowed the captured images to be transformed to a common reference frame, while the control beads allowed the calculation of the foci (i.e., the x-ray sources) locations to enable the analysis. The images were captured on digital detector plates (4343CB; Varian Medical Systems, Palo Alto, Calif., USA) as greyscale images with relative intensity values in standard medical DICOM format. The overlap of the two radiography systems' fields of view made up the 3D viewing volume.

### The Spine Positioning Device and Image Data Acquisition

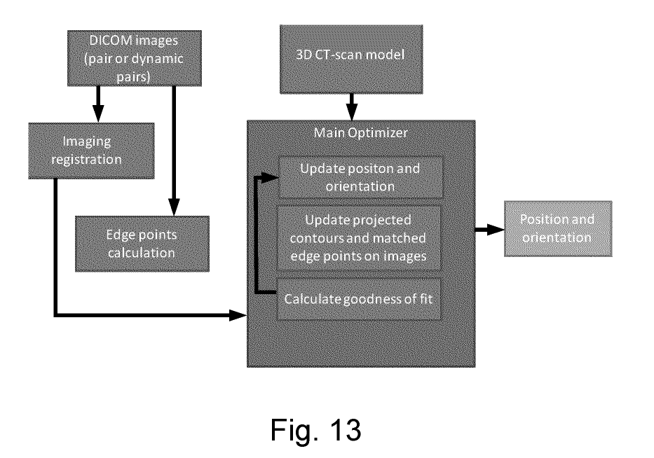

For each of the image sequence recordings, the patient was instructed on the posture and motions to be used during imaging. In order to keep a patient's spine within the 3D viewing volume of the stereo radiography system during the imaging process, the patient was positioned in the positioning device (similar to that exemplified in FIGS. 4(A), 4(B). For some of the imaging sequences, the patient stood with their feet positioned toward the rear of the platform, their knees rested on the knee support, and with their pelvis rested against the pelvic support while a technician monitored their positioning, posture, and the patient moved from a neutral position to flexion then to extension and then back to the neutral position. For other imaging exposures, the patient sat on the edge of the imaging table and held a neutral position followed by a supine position with the knees flexed. Each of the image sequence recordings was reviewed by the technologist to ensure image quality and the regions of interest were captured. The images were then transferred using tele-radiology technology to the image analysis center for analysis following the process outlined in the exemplary flow chart shown in FIG. 13.

### System Configuration Determination

The radiographic images were loaded onto a computer system for calculation of the parameters that described the detailed configuration of the imaging system. The fiducial beads in the reference box were located in the images and their locations tabulated. Based on the known measured locations of these beads, a projective transformation was calculated that matched the bead locations to the tabulated locations from the images. The control beads of the reference box were located in the images and their locations tabulated. Based on the known measured locations of the fiduciary beads and the control beads, the locations of the two foci were calculated.

### Reconstruction of Vertebral Pose and Orientation

The 3D shapes of the vertebrae were represented by triangulated meshes reconstructed from CT scans previously acquired from the patient. The location of the foci and the parameters describing the projective transform were used to calculate the projected contours onto the fiducial plane for any given position and orientation of each vertebra. A graphic user interface allowed the operator to manipulate the position and orientation via sliders, and to immediately see the results of the projected contours onto the radiographic image. In this way, the operator set the initial position and orientation, which were saved and used as the starting points for the optimizer.

An objective function was made available to the optimizer which calculated a goodness-of-fit score between the projected contours and selected image edges given a position and orientation. The detected contours were determined based on edge detection on the image using a Canny filter. The goodness of fit score was based on a modified Hausdorff Distance.

The optimizer used the objective function to find the pose and orientation that provided the best fit to the radiographic images, within a predefined search space. In this example, the optimizer first used Scatter Search Optimization as a global optimization method generally following the process illustrated in FIG. 13. A second round of optimization attempted to further increase the goodness-of-fit with a local-gradient-based optimizer. The initial starting estimates were uniformally distributed along the predefined search space and centered on the user initialized estimates. The optimizer returned the final position and orientation of the 3D vertebra model.

In the same way, the final position and orientation of the 3D vertebra was calculated for every set of images in a series. The optimizer used the position and orientation of a previous image in a series, combined with knowledge of the context of the acquisition to automatically initialize the position and orientation without user interaction. In this way, the vertebrae poses of the target region were reconstructed for the entire set of multi-frame radiographic images throughout the motion.

### Presentation of Reconstructed 3D Motion

With the final position and orientation of the 3D vertebra models determined for every set in a series, the reconstructed 3D motion was available for presentation. The data was presented via a specialized app which connected with the database server to retrieve the analysis results. A time-series of 3D data could be navigated via a slider or with movement of the cursor over the viewing area, or could be viewed with a continuous dynamic loop. The frame of reference of the motion could be set by the user to any of the vertebral segments of interest or to a static global reference frame. The user could change the viewing angle of the 3D models to achieve any viewing angle. Also, the user could select the shading and transparency of each vertebral segment. Based on diagnostic measurements relevant to the reconstructed 3D motion, color coding was used to highlight those segments which deviated from known normative motion. The color presented was based on color mapping indicative to the degree or grade of deviation from known normative motion.

### Calculation and Presentation of Vertebral Morphology

A statistical shape model was fit in 3D to the CT-based mesh model of the vertebra of interest using a Particle Swarm Optimization after an initial alignment using an iterative closest point algorithm. The modes of shape variations described the morphological relationship between the patient's vertebra and the normative data contained in the statistical shape model. The patient's 3D vertebra models were then presented in their own visualization with color mapping indicative of these morphological differences. The user could select which modes of variation (or combination thereof) to select for this visualization. Known combinations established from normative data were also available as presets and available for visualization.

1. A diagnostic method for quantitatively measuring spinal instability in a patient, the method comprising:
capturing a series of multi-frame stereo radiographic images of a target region of the patient's spine, wherein the patient is moving through a range of motion that allow for motion of vertebrae in the target region of the spine to be captured in the series of multi-frame stereo radiographic images;
reconstructing a three-dimensional model of the target region of the patient's spine moving through the range of motion, wherein a relative three-dimensional position and orientation for each vertebra in the target region is calculated based on the radiographic images for each frame of the series of images; and
measuring a change in the relative three-dimensional position and orientation of each vertebra in the three-dimensional model of the target region throughout the motion, wherein the measured change reflects the amount of spinal instability in the patient.

2. The method according to claim 1, additionally comprising:
displaying the change in the relative three-dimensional position and orientation of each vertebra as a three-dimensional movie.

3. The method according to claim 1, additionally comprising:
determining and analyzing the shape of the vertebrae.

4. The method according to claim 1, wherein calculation of the relative three-dimensional position and orientation for each vertebra in the target region in step (b) is by iterative optimization based on the radiographic images for each frame of the series of images.

5. The method according to claim 1, wherein the change in the relative three-dimensional position and orientation of each vertebra is displayed in a series of sequential digital images.

6. The method according to claim 1, wherein the change in the relative three-dimensional position and orientation of each vertebra is displayed in a dynamic bar graph.

7. A method for assessing a patient's suitability for an orthopaedic procedure, the method comprising:
capturing a series of multi-frame stereo radiographic images of a target region of the patient's spine, wherein the patient is moving through a range of motion that allow for motion of vertebrae in the target region of the spine to be captured in the series of multi-frame stereo radiographic images;
reconstructing a three-dimensional model of the target region of the patient's spine moving through the range of motion, wherein a relative three-dimensional position and orientation for each vertebra in the target region is calculated based on the radiographic images for each frame of the series of images;
measuring a change in the relative three-dimensional position and orientation of each vertebra in the three-dimensional model of the target region throughout the motion; and
comparing the measured change in the three-dimensional model to instability data standards for normative and varying levels of instability, wherein the comparison indicates the degree of instability and the patient's suitability for an orthopaedic procedure.

8. The method according to claim 5, wherein calculation of the relative three-dimensional position and orientation for each vertebra in the target region in step (b) is by iterative optimization based on the radiographic images for each frame of the series of images.

9. The method according to claim 7, wherein step (b) further comprises determining the shape of the vertebrae and step (d) also further comprises comparing the shape of the vertebrae to normative shapes and shapes of patients with spinal pathology, wherein the comparison indicates the degree of pathology and the patient's suitability for an orthopaedic procedure.

10. The method according to claim 7, further comprising:
classifying the measured change by type and degree of instability of the vertebrae to determine the suitability of the patient for the orthopaedic procedure.

11. The method according to claim 7, wherein the change in the relative three-dimensional position and orientation of each vertebra is displayed in a series of sequential digital images.

12. The method according to claim 7, wherein the change in the relative three-dimensional position and orientation of each vertebra is displayed in a dynamic bar graph.

13. The method according to claim 7, wherein the orthopaedic procedure is spinal fusion, artificial disk replacement, dynamic stabilization procedures or conservative treatment.

14. A radiographic imaging method for generating a three-dimensional reconstruction of the movement of a target region of a patient's spine, the method comprising:
capturing a series of multi-frame radiographic images of the target region of the patient's spine, the radiographic images comprising a pair of images taken at an angle of each other to capture images within a viewing volume wherein the patient is moving through a range of motion;
calculating foci and edge data of vertebrae captured in a radiographic image in the series and consolidating the data to a common reference frame;
determining a general three-dimensional position and orientation of the vertebrae;
iteratively manipulating the general three-dimensional position and orientation of the vertebrae against the data in the common reference frame to achieve a best-fit three-dimensional position and orientation for each vertebra in the radiographic image; and
repeating steps b to d for each image pair of a series;
wherein a three-dimensional model of the target region of the patient's spine moving through the range of motion is generated.

15. The method according to claim 14, wherein step (c) involves using population-based vertebral shape models.

16. The method according to claim 15, wherein the population-based vertebral shape model is a statistical shape model.

17. The method according to claim 14, wherein step (c) further comprises determining shape of the vertebrae.

18. The method according to claim 14, wherein step (c) is derived from a three-dimensional model of the patient's vertebral spine.

19. The method according to claim 14, wherein the three-dimensional model of the patient's vertebral spine is created from a CT-scan or an MRI of the patient's spine.

20. The method according to claim 14, wherein the change in the relative three-dimensional position and orientation of each vertebra is displayed in a series of sequential digital images.

21. The method according to claim 14, wherein the change in the relative three-dimensional position and orientation of each vertebra is displayed in a dynamic bar graph.

22. A positioning apparatus for maintaining the position of a patient in a viewing area during radiographic imaging throughout a series of patient movements, the apparatus comprising:
a base for supporting a foot platform on which the patient stands when in position for radiographic imaging, the foot platform having a front end and a rear end; and
a pelvic support extending from the base above the foot platform at the rear end, the pelvic support configured to support the patient's pelvis.

23. The positioning apparatus according to claim 22, wherein the series of patient movements comprises lumbar flexion and extension.

24. The positioning apparatus according to claim 22, further comprising a knee support extending from the base above the foot platform at the front end, the knee support configured to support the patient's knees when the patient is positioned with ankles, knees and hips flexed.
